(19) United States
(12) Patent Application Publication
(10) Pub. No.: US 2013/0274581 A1
(43) Pub. Date: Oct. 17, 2013

(54) BLOOD GLUCOSE MEASUREMENT MODULE, SMART PHONE COMBINABLE WITH THE BLOOD GLUCOSE MEASUREMENT MODULE, AND BLOOD GLUCOSE MEASUREMENT METHOD USING THE SAME

(71) Applicants: In Hwan Choi, Suwon-si (KR); Jeong In Bag, Anyang-si (KR); Ji Hoon Pyo, Seoul (KR)

(72) Inventors: In Hwan Choi, Suwon-si (KR); Jeong In Bag, Anyang-si (KR); Ji Hoon Pyo, Seoul (KR)

(73) Assignee: PHILOSYS CO., LTD., Funsan-si, Jeollabuk-do (KR)

(21) Appl. No.: 13/995,844

(22) PCT Filed: Apr. 13, 2011
(86) PCT No.: PCT/KR2011/002629

§ 371 Date: Jun. 19, 2013

(30) Foreign Application Priority Data
Dec. 31, 2010 (KR) 10-2010-0139826

(51) Int. Cl. A61B 5/145; A61B 5/00

(52) U.S. Cl. CPC A61B 5/14532; A61B 5/742
USPC 600/365

(57) ABSTRACT

Disclosed herein are a blood glucose measurement module, a smart phone combinable with the blood glucose measurement module, and a blood glucose measurement method using the same. The blood glucose measurement module includes a body, a strip reception slit, and a plug. The body includes a blood glucose measurement unit for measuring a blood glucose level of blood absorbed into a test strip and a CPU for calculating and transmitting the results of the measurement of the blood glucose level. The strip reception slit is formed in a part of the body, and receives the test strip. The plug is formed on one end surface of the body, and is easily fitted into the socket of a smart phone.

### TECHNICAL FIELD

The present invention relates, in general, to a blood glucose measurement module, a smart phone combinable with the blood glucose measurement module, and a blood glucose measurement method using the same, and, more particularly, to a blood glucose measurement module that is capable of forming a blood glucose meter in combination with a smart phone by using the display and application of the smart phone, a smart phone combinable with the blood glucose measurement module, and a blood glucose measurement method using the same.

### BACKGROUND ART

A blood glucose meter is used to measure the blood glucose level of a diabetic. A blood glucose meter measures the blood glucose level using a mechanism of converting electrons, which are generated in the process in which hydrogen peroxide (generated by the oxidation of glucose in the blood by glucose oxidase) is converted into oxygen, into a current signal using electrodes, quantifying the current signal, and measuring the blood glucose level.

Accordingly, a blood glucose meter includes a blood glucose measurement unit for measuring a blood glucose level and checking a variation in the blood glucose level, a power supply unit, a current sensor, a memory unit, and a display unit. Recently, a method is being widely used in which, using a test strip, a patient collects his or her blood, applies the blood to a blood glucose meter, and checks and manages the results of the measurement of the blood glucose level.

However, such conventional blood glucose meters have the problems of being inconvenient to carry because they are separately formed, having small displays, and having a very narrow range of internal use of measured data. Although a product having a function of transmitting measured data has been developed so as to overcome the above problems, the product has the disadvantages of being incompatible with portable electronic devices such as a smart phone and having poor commercial applicability because data cannot be easily transmitted. Above all, the product causes inconvenience to the user of a smart phone because the user should carry both the smart phone and the blood glucose meter in order to use the blood glucose meter.

In order to overcome the above problems, Korean Unexamined Patent Application Publication No. 2003-0096652 entitled “Blood Glucose Meter and Method using Mobile Phone” discloses a blood glucose meter using a mobile phone, which includes a power supply unit configured to supply power voltage; a current sensor configured to have two terminals, detect a variation in amount of current corresponding to the concentration of analysis material between the two terminals, and convert a detected signal into a digital signal; a Mobile Station Modem (MSM) configured to receive the digital signal from the current sensor, receive a data table by outputting a control signal, and acquire data about the concentration of the material analyzed based on the digital signal; a memory unit configured to store the data table indicating current and voltage relationships based on the reagent code, and output the data table to the MSM in response to the control signal from the MSM; and an LCD display unit configured to receive and display the data about the concentration from the MSM. However, although this conventional technology is configured such that a blood glucose measurement function is contained in an existing mobile phone and has the advantage of being convenient to carry because it is not necessary to carry a separate blood glucose meter, the conventional technology has the problems of requiring a separate manufacturing apparatus for separately manufacturing only mobile phones having a blood glucose measurement function, having a narrow range of use of measured data, and experiencing failures when the mobile phone is used to measure blood glucose.

In order to solve these problems, there is a need for the development of a module-type blood glucose meter which can take advantage of the power, application and display of a smart phone and which can be externally combined with the start phone. A smart phone is an intelligent terminal in which computer support functions such as Internet communication and information searching have been added to a mobile phone, and is a portable communication device on which a user can install desired applications. Accordingly, when a blood glucose measurement module having the minimum number of elements required to measure and calculate the blood glucose level is combined with and then used in conjunction with a smart phone, the problems of the above-described conventional technology can be considerably surmounted.

Accordingly, the inventors of the present invention conceived of a blood glucose measurement module that can be mass produced at low cost because it can be combined with and used in conjunction with the smart phone without requiring a separate display and a separate power supply unit, that is lightweight and small in size, that can diversify the use of data using a variety of types of computer service functions such as Internet communication and information searching because the blood glucose measurement module can be used for an application of a smart phone, that is convenient to carry, and that can prevent the smart phone from being contaminated or damaged by blood, a smart phone combinable with the blood glucose measurement module, and a blood glucose measurement method using the same.

### DISCLOSURE OF INVENTION

### Technical Problem

Accordingly, the present invention has been made keeping in mind the above problems occurring in the prior art, and an object of the present invention is to provide a blood glucose measurement module that is capable of being mass produced at low cost because it can be combined with and then used in conjunction with a smart phone without requiring a separate display and a separate power supply unit, a smart phone combinable with the blood glucose measurement module, and a blood glucose measurement method using the same.

Another object of the present invention is to provide a blood glucose measurement module that is lightweight and small in size and is convenient to carry, a smart phone combinable with the blood glucose measurement module, and a blood glucose measurement method using the same.

A further object of the present invention is to provide a blood glucose measurement module that is capable of diversifying the use of data using a variety of types of computer service functions such as Internet communication and information searching because it can be used with an application of a smart phone, a smart phone combinable with the blood glucose measurement module, and a blood glucose measurement method using the same.

### Solution to Problem

In order to accomplish the above objects, the present invention provides a blood glucose measurement module, including a body configured to comprise a blood glucose measurement unit for measuring a blood glucose level of blood absorbed into a test strip and a CPU for calculating and transmitting results of the measurement of the blood glucose level; a strip reception slit formed in a part of the body, and configured to receive the test strip; and a plug formed on one end surface of the body, and configured to be easily fitted into the socket of a smart phone.

The plug may be configured in the form of a phone jack plug or a pin plug.

In order to accomplish the above objects, the present invention provides a smart phone having a display unit and a power supply unit, including a socket that can be combined with a plug of the blood glucose measurement module.

The socket may a phone jack socket combinable with a phone jack plug or a port combinable with a pin plug.

The smart phone may further include an application program capable of processing and managing data about measured blood glucose levels.

In order to accomplish the above objects, the present invention provides a blood glucose measurement method using a smart phone combinable with a blood glucose measurement module, including fitting a plug of the blood glucose measurement module into the socket of the smart phone; turning on power of the blood glucose measurement module using power of the smart phone and performing an internal test; if no abnormality is found in the internal test, inserting a test strip into a strip reception slit of the blood glucose measurement module and checking a change in the test strip; applying blood to the test strip and calculating results of the measurement of the blood glucose level of the blood; and storing the results of the measurement.

The blood glucose measurement method may further include, before the fitting, executing a blood glucose measurement application of the smart phone.

The inserting may include displaying on the smart phone a screen indicating the insertion of the test strip; the applying may include sequentially displaying on the smart phone a screen indicating the application of the blood and a screen indicating that the blood glucose level is being measured; and the storing may include displaying the results of the measurement of the blood glucose level on the smart phone.

### Advantageous Effects of Invention

The present invention provides the advantageous effects of being mass produced at low cost because the blood glucose measurement module can be combined with and then used in conjunction with the smart phone without requiring a separate display and a separate power supply unit, being lightweight and small in size, diversifying the use of data using a variety of types of computer service functions such as Internet communication and information searching because the blood glucose measurement module can be used for an application of a smart phone, being convenient to carry, and preventing the smart phone from being contaminated or damaged by blood.

### BRIEF DESCRIPTION OF DRAWINGS

The above and other objects, features and advantages of the present invention will be more clearly understood from the following detailed description taken in conjunction with the accompanying drawings, in which:

FIG. 1 is a perspective view schematically showing the appearance of a blood glucose measurement module according to the present invention;

FIG. 2 is a perspective view schematically showing fitting the blood glucose measurement module of the present invention into a smart phone;

FIG. 3 is a flowchart schematically showing a blood glucose measurement method using a smart phone which can be combined with the blood glucose measurement module according to the present invention; and

FIG. 4 is a flowchart showing a blood glucose measurement method using the blood glucose measurement module and a smart phone according to an embodiment of the present invention.

### BEST MODE FOR CARRYING OUT THE INVENTION

Reference should now be made to the drawings, in which the same reference numerals are used throughout the different drawings to designate the same or similar components.

A blood glucose measurement module, a smart phone which can be combined with the same, and a blood glucose measurement method using the same according to embodiments of the present invention will be described in detail below with reference to the accompanying drawings.

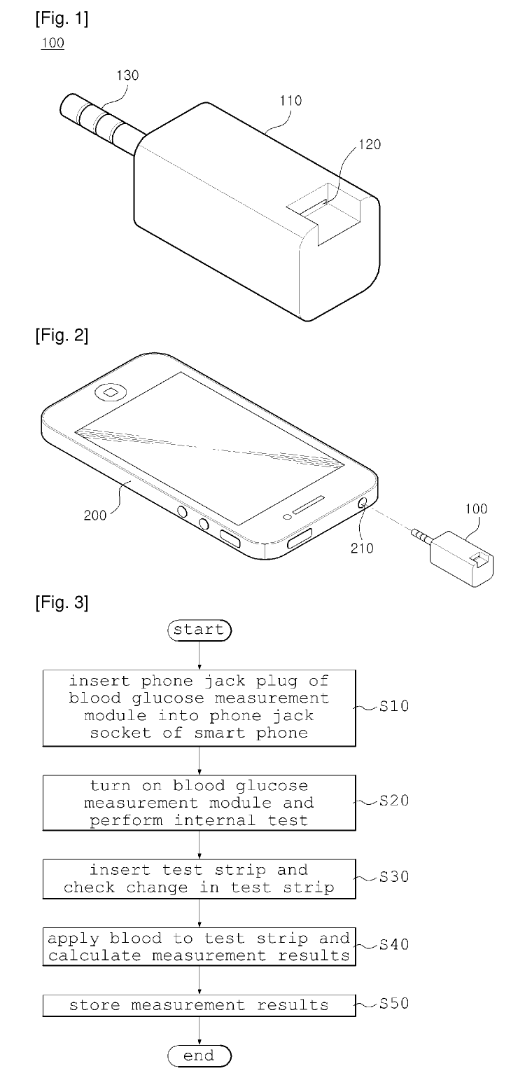

FIG. 1 is a perspective view schematically showing the appearance of a blood glucose measurement module according to the present invention.

As shown in FIG. 1, a blood glucose measurement module 100 according to the present invention includes a body 110, a strip reception slit 120, and a plug 130.

The body 110 contains units for measuring the blood glucose level, including a blood glucose measurement unit for measuring the blood glucose level of blood absorbed into a test strip and a Central Processing Unit (CPU) for calculating and transmitting the results of the measurement of the blood glucose level, and has the advantages of being small in size and lightweight because the body 110 does not need to include a power supply unit and a display unit.

The strip reception slit 120 is formed in a part of the body 110, is configured to receive a test strip, and has no special limitations imposed on its structure and design so long as the structure and the design allow a test strip to be easily inserted thereinto.

Furthermore, the plug 130 is formed on one end surface of the body 110, and is configured to be easily fitted into the socket of a smart phone. Although the type and shape of the plug 130 are not limited to a specific type and shape, it is preferred that the plug 130 be configured in the form of a phone jack plug or a pin plug in order to allow the plug 130 to be easily fitted into a smart phone.

FIG. 2 is a perspective view schematically showing fitting the blood glucose measurement module 100 of the present invention into a smart phone 200.

As shown in FIG. 2, the blood glucose measurement module 100 according to the present invention may be fitted into the socket 210 of the smart phone 200, and then be used in this state. That is, when the blood glucose measurement module 100 is used with the plug of the blood glucose measurement module 100 fitted into the socket 210 of the smart phone 200, the blood glucose measurement module 100 obtains blood glucose data using a method of measuring and calculating the blood glucose level of blood absorbed into a test strip using power supplied by the power supply unit of the smart phone 200 and transmitting data about the measured blood glucose level to the smart phone 200. In this process, it is preferable to use a blood glucose measuring application program of the smart phone 200 that is capable of processing and managing data about the measured blood glucose levels.

The case where the blood glucose measurement module 100 is used with the plug of the blood glucose measurement module 100 fitted into the socket 210 of the smart phone 200 has the advantage of excellent commercial applicability because input, output and power can all be used. Furthermore, although it is preferable to use Frequency Shift Keying (FSK) as a data communication method, the data communication method is not limited thereto, but may vary depending on the purpose of use and means used.

Although the type and shape of the socket 210 are not limited to a specific type and shape, it is preferable to configure the socket 210 in the form of a phone jack socket combinable with the phone jack plug of the blood glucose measurement module 100 or in the form of a port combinable with a pin plug. However, the combining structure is not limited to the above-described combining structure because the components of the structure may be formed in the smart phone and the blood glucose measurement module in the opposite manner, structures which can be easily conceived of by those skilled in the art based on replacement and modification pertain to the scope of the present invention within the range that does not depart from the scope and spirit of the invention.

FIG. 3 is a flowchart schematically showing a blood glucose measurement method using a smart phone which can be combined with the blood glucose measurement module according to the present invention.

As shown in FIG. 3, the blood glucose measurement method using a smart phone combinable with the blood glucose measurement module according to the present invention includes step S10 of fitting the plug of the blood glucose measurement module into the socket of the smart phone; step S20 of turning on the power of the blood glucose measurement module using the power of the smart phone and performing an internal test; step S30 of, if no abnormality is found in the internal test, inserting a test strip into the strip reception slit of the blood glucose measurement module and checking a change in the test strip; step S40 of applying blood to the test strip and calculating the results of the measurement of the blood glucose level of the blood; and step S50 of storing the results of the measurement.

Step S20 of turning on the power of the blood glucose measurement module using the power of the smart phone includes a process in which the blood glucose measurement module receives a voltage signal from the amplifier of the smart phone and converts the voltage signal into a stable voltage.

Furthermore, the blood glucose measurement method according to the present invention may further include, before step S10, the step of executing a blood glucose measurement application of the smart phone, and steps S30 to S50 may include the steps of utilizing the application program, respectively. For example, step S30 includes displaying on the smart phone a screen indicating the insertion of the test strip, step S40 includes sequentially displaying on the smart phone a screen indicating the application of the blood and a screen indicating that blood glucose level is being measured, and step S50 includes displaying the results of the measurement of the blood glucose level on the smart phone.

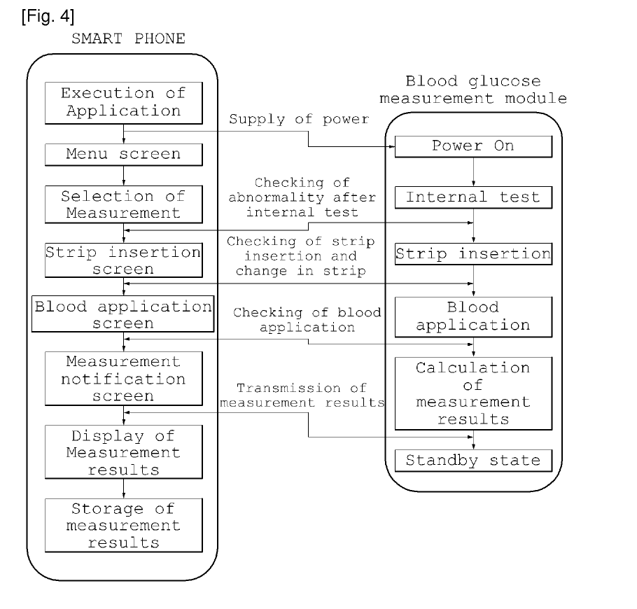

FIG. 4 is a flowchart showing a blood glucose measurement method using the blood glucose measurement module and a smart phone according to an embodiment of the present invention.

As shown in FIG. 4, the blood glucose measurement module according to the embodiment of the present invention may be combined with and then used in conjunction with the smart phone.

First, the application of the smart phone is executed, and “measurement” is selected from a menu screen. Furthermore, the blood glucose measurement module receives power from the power supply unit of the smart phone, conducts an internal test, and checks whether there is no abnormality based on the results of the internal test. Thereafter, the test strip is inserted into the blood glucose measurement module in accordance with a screen indicating the insertion of the test strip, and a change in the test strip is checked. In this process, a screen showing the results of the checking of the change in the test strip may be displayed after the screen indicating the insertion of the test strip has been displayed. Thereafter, when blood is applied to the test strip in accordance with the screen indicating the application of blood and the application of the blood is detected, a screen indicating that the blood glucose is being measured is displayed on the smart phone. After the blood glucose has been measured, the blood glucose measurement module calculates and transmits the results of the measurement, the smart phone displays and stores data about the results of the measurement, and the blood glucose measurement module enters a standby state, thereby completing the overall process.

Although the preferred embodiments of the present invention have been disclosed for illustrative purposes, those skilled in the art will appreciate that various modifications, additions and substitutions are possible without departing from the scope and spirit of the invention as disclosed in the accompanying claims.

### INDUSTRIAL APPLICABILITY

The blood glucose measurement module according to the present invention has the advantages of being combined with a smart phone and then being used as a part of a blood glucose meter, being lightweight and small in size, and preventing the smart phone from being contaminated by blood. Furthermore, the blood glucose measurement module has the advantage of having commercial applicability superior to that of a conventional blood glucose meter because data about blood glucose transmitted to and stored in the mobile phone may be used in various ways by means of the various functions of the smart phone.

1. A blood glucose measurement module, comprising:
a body configured to comprise a blood glucose measurement unit for measuring a blood glucose level of blood absorbed into a test strip and a Central Processing Unit (CPU) for calculating and transmitting results of the measurement of the blood glucose level;
a strip reception slit formed in a part of the body, and configured to receive the test strip; and
a plug formed on one end surface of the body, and configured to be easily fitted into a socket of a smart phone.

2. The blood glucose measurement module according to claim 1, wherein the plug is configured in a form of a phone jack plug or a pin plug.

3. A smart phone having a display unit and a power supply unit, comprising a socket that can be combined with a plug of a blood glucose measurement module set forth in claim 1.

4. The smart phone according to claim 3, wherein the socket is a phone jack socket combinable with a phone jack plug or a port combinable with a pin plug.

5. The smart phone according to claim 3, further comprising an application program capable of processing and managing data about measured blood glucose levels.

6. A blood glucose measurement method using a smart phone combinable with a blood glucose measurement module, comprising:
fitting a plug of the blood glucose measurement module into the socket of the smart phone;
turning on power of the blood glucose measurement module using power of the smart phone and performing an internal test;
if no abnormality is found in the internal test, inserting a test strip into a strip reception slit of the blood glucose measurement module and checking a change in the test strip;
applying blood to the test strip and calculating results of the measurement of the blood glucose level of the blood; and
storing the results of the measurement.

7. The blood glucose measurement method according to claim 6, further comprising, before the fitting, executing a blood glucose measurement application of the smart phone.

8. The blood glucose measurement method according to claim 6, wherein:
the inserting comprises displaying on the smart phone a screen indicating the insertion of the test strip;
the applying comprises sequentially displaying on the smart phone a screen indicating the application of the blood and a screen indicating that the blood glucose level is being measured; and
the storing comprises displaying the results of the measurement of the blood glucose level on the smart phone.
